(19) United States
(12) Patent Application Publication
(10) Pub. No.: US 2026/0035754 A1
(43) Pub. Date: Feb. 5, 2026

(54) HOLISTIC CHARACTERIZATION OF BIO-MARKER INDICATORS FOR MICROBIAL INFLUENCED CORROSION IN HYDROCARBON RESERVOIR

(71) Applicant: SAUDI ARABIAN OIL COMPANY, Dhahran (SA)

(72) Inventors: Abdulmohsen A. AL-HUMAM, Dhahran (SA); Manar M. ALAHMARI, Dhahran (SA)

(73) Assignee: SAUDI ARABIAN OIL COMPANY, Dhahran (SA)

(21) Appl. No.: 18/790,884

(22) Filed: Jul. 31, 2024

(51) Int. Cl. C12Q 1/689; C12Q 1/6874; G16B 20/00; G16B 40/20

(52) U.S. Cl. CPC C12Q 1/689; C12Q 1/6874; G16B 20/00; G16B 40/20

(57) ABSTRACT

Methods described herein include machine-learning based algorithms identifying and predicting bio-markers associated with microbial-influenced corrosion. Methods may comprise: performing genomic analysis on a genomic material of a collected sample based on metagenomics sequencing to provide sequenced genomic data; clustering the sequenced genomic data to form sequenced genomic clusters; wherein the sequenced genomic clusters represent a predictive function provided by the sequenced genomic data to a trained machine learning (M.L.) predictive algorithm; sorting the sequenced genomic clusters into one or more microbial influenced corrosion-based categories, wherein the one or more microbial influenced corrosion-based categories are based on one or more microbial influenced corrosion-related microorganisms or a compound of the related microorganisms; and characterizing a corrosion biomarker of the one or more microbial influenced corrosion-based categories, wherein the corrosion biomarker is logically linked with an increased likelihood of microbial influenced corrosion based on the predictive function of the sequenced genomic clusters.

### FIELD OF THE DISCLOSURE

Systems and methods for the development of metabolic biomarkers linked to microbial contamination in oil and gas fields.

### BACKGROUND OF THE DISCLOSURE

Oilfield production requires fine machinery both above and below ground to function at optimal conditions for both the exploration and extraction of oil reserves. These equipment are costly to maintain, or replace in cases of breakdown. One of the leading causes for equipment degradation and breakdown has been linked to microbial influenced corrosion (MIC), where single celled or multi-cellular microbial organisms such as bacterium contaminate the oilfield site by attaching to metal surfaces and forming biofilms. These biofilms can initiate the corrosion process in a variety of ways, including by oxidation, altering pH, and producing acids. MIC involve a broad spectrum of bacterium, with sulfur-reducing bacteria (SRB) as a lead cause of electrochemical corrosion. MIC bacterium sustain growth from organic and inorganic nutrients, and have been proven hard to detect at oil and gas reservoirs before contamination causes extensive and sometimes permanent damage on pipeline systems.

Current methods for MIC monitoring and oil and gas production sites involve slow culture-growing assays such as the Most Probable Number (MPN). These assays can take up to two weeks to grow. Once the sample has been grown and quantified by MPN, testing for metabolites such as ATP or other bacterial byproducts determine the quantity and veracity of contamination at the test site. These assays are limited in scope of the organisms they can detect that may be indicators of MIC, as approximately less than 10% of corrosion causing bacterium are detectable by MPN. Currently, there is no explicit characterizing methods for decisive treatment methodology. This leaves hydrocarbon production sites vulnerable for longer periods of time.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In various embodiments, methods of the present disclosure comprise performing genomic analysis on a genomic material of a collected sample based on metagenomics sequencing to provide sequenced genomic data; clustering the sequenced genomic data to form sequenced genomic clusters; wherein the sequenced genomic clusters represent a predictive function provided by the sequenced genomic data to a trained machine learning (M.L.) predictive algorithm; sorting the sequenced genomic clusters into one or more microbial influenced corrosion-based categories, wherein the one or more microbial influenced corrosion-based categories are based on one or more microbial influenced corrosion-related microorganisms or a compound of the related microorganisms; and characterizing a corrosion biomarker of the one or more microbial influenced corrosion-based categories, wherein the corrosion biomarker is logically linked with an increased likelihood of microbial influenced corrosion based on the predictive function of the sequenced genomic clusters.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is a block diagram of a non-limiting system and method of characterizing bio-markers for microbial influenced corrosion from a collected sample via 16S amplicon sequencing.

FIG. 2 is a block diagram of a non-limiting system and method of characterizing bio-markers for microbial influenced corrosion from a collected sample via shotgun whole metagenome sequencing.

FIG. 3 is a block diagram of a non-limiting system and method of characterizing bio-markers for microbial influenced corrosion from a collected sample via both 16S amplicon genetic sequencing and shotgun whole metagenome sequencing.

FIG. 4 is a schematic of classes of microbial influenced corrosion causing organisms and associated metabolic byproducts.

### DETAILED DESCRIPTION

Embodiments in accordance with the present disclosure generally relate to systems and methods for early detection of microbial contamination in a hydrocarbon reservoir environment; in particular, the present disclosure relates to methods for broad spectrum analysis and characterization of bio-marker indicators of microbial influenced corrosion (MIC) organisms.

Metal oil and gas pipeline and machinery degradation onset by water-borne microbial contamination is a major limiting factor in hydrocarbon discovery and recovery. Recently, this issue has been accelerated with techniques such as water injection treatments and currently accounts for approximately 20% of corrosion costs. Unfortunately, early detection of these MIC organisms by current standard tools and methods is laborious and limited in range of detectable organisms. The NACE TMO194 standard, which utilizes the most probable number (MPN) culture growth assay, simply quantifies the microbial population of a sample, and can easily overlook a variety of sulfate-reducing prokaryotes such as iron-reducing bacteria (FeRB), sulfate-reducing bacteria (SRB), methanogenic archaea (MA), fermentative bacteria (FB), denitrifying bacteria (DEN), and the like. Furthermore, identifying microbial contamination is time-sensitive, with corrosion worsening as bacterium grow exponentially. Current standard protocols such as MPN can take anywhere from about two to four weeks for results to return once a sample is collected.

More rapid detection methods may focus more on metabolic markers for identifying a microbial population within hydrocarbon reservoirs. The RapidChek®II method is a metabolic detector of SRB microbes in a sample by the quantification of adenosine-5-phosphosulfonate (APS) reductase enzyme via interaction with the color indicator chromagen. This metabolic test may take up to one hour to complete, and gives an estimate of the SRB population within the sample, but can only detect limited types of corrosion-causing microbes.

Genetic MIC screening expands the scope of microbial targets detected within a sample. The most common genetic screening for MIC is the Next-Generation Sequencing (NGS) 16S amplicon method, which amplifies the conserved and variable regions that are abundant in bacterial and archaca genetic material (genes). However, 16S suffers from low resolution between closely related species, and also overlooks microbes in low abundance, therefore failing to capture the whole picture of the microbial community in a sample. Recent utilization of molecular methods to quantify and identify microbial populations more comprehensively may include use of quantitative polymerase chain reaction (qPCR) testing. Once genes are extracted and purified, qPCR quantifies specific genes known to affect MIC metabolism. However, without methods to identify and cluster the generated data, it may be difficult to distinguish all MIC related microbes, or elucidate new bio-markers of corrosion.

Current treatments for MIC may either involve physical removal and cleaning of corrupted equipment, or chemical treatment with broad-spectrum and expensive biocides. These solutions may be moot if the contamination has spread undeterred for a prolonged period of time.

Accordingly, systems and methods of the present disclosure may comprise: performing genomic analysis on a genomic material of a collected sample based on metagenomics sequencing to provide sequenced genomic data; clustering the sequenced genomic data to form sequenced genomic clusters; wherein the sequenced genomic clusters represent a predictive function provided by the sequenced genomic data to a trained machine learning (M.L.) predictive algorithm; sorting the sequenced genomic clusters into one or more microbial influenced corrosion-based categories, wherein the one or more microbial influenced corrosion-based categories are based on one or more microbial influenced corrosion-related microorganisms or a compound of the related microorganism; and characterizing a corrosion biomarker of the one or more microbial influenced corrosion-based categories, wherein the corrosion biomarker is logically linked with an increased likelihood of microbial influenced corrosion based on the predictive function of the sequenced genomic clusters.

### Definitions

As used herein, the term “hydrocarbon reservoir,” and grammatical variants thereof, refers generally to a subterranean or subsurface formation comprising pores that may contain a hydrocarbon (e.g., oil, gas, the like) as well as associated water, brine, and the like within aforementioned pores. The hydrocarbon reservoir may be comprised of a plurality of layers of rock.

As described herein, the term “corrosive agent,” “caustic chemicals,” and grammatical variants thereof, refers generally to a material that may have the capacity to damaging or disintegrate another material once surface contact has been established. The corrosive agent or caustic chemical may be in any form of matter such as a solid, liquid, or gas. This surface contact may initiate a chemical reaction. Examples of corrosive agents may include oxygen, hydrogen sulfide, carbon dioxide, and their like. Effects of corrosive agents may include, but are not limited to, localized corrosion, pitting, softening of a surface, uneven surface roughness, the like, or any combination thereof.

As described herein, the term “electrochemical corrosion,” and grammatical variants thereof, refers generally to a spontaneous natural reaction that chemically stabilizes a metal and impairs function. The stable metal form may present as an oxide, hydroxide, sulfide, or the like. The reaction may occur as an enzymatic process, involving an enzymatic electron-transfer process, wherein an anodic reaction of ionization or oxidation occurs on the metal surface, releasing an electron and creating metal ions. The electron is then received by a cathodic reaction known as reduction, wherein chemical compounds accept the electron. Electrochemical corrosion may take place in an environment that comprises oxygen and water such as moisture.

As described herein, the term “microbial influenced corrosion (MIC),” “bio-corrosion,” “bio-degradation,” and grammatical variants thereof, refers the influence of microbial organisms in the corrosion kinetics of metal and nonmetal surfaces. This process is biological in nature, and initiated by microorganisms forming biofilms and releasing metabolic byproducts initiating electrochemical reactions. These microorganisms may comprise microscopic organisms that may be single celled or multicellular, and can be classified as Prokaryotes comprising of the Domains Archaca and Bacteria. This process may result in the degradation of metal, alloy, or plastic surfaces, as well as protective coatings, emulsions, and oils.

As used herein, the term “genomic material,” “gene,” “genetic material,” “DNA,” “RNA,” and grammatical variants thereof, refers generally to genomic material comprising biopolymers composed of nucleotide base pairs that are constructed in a double-stranded helix formation. This may be found in the nucleus and or cytoplasm of an organism. This biopolymer is responsible for relaying genetic information in the cell, and may direct the expression or depression of other genes.

As used herein, the term “rRNA” and grammatical variants thereof, refers generally to a biopolymer composition comprising of ribosomal ribonucleic acids (rRNA).

As used herein, the term “sequencing” and grammatical variants thereof, refers generally to the reading and outputting of the order sequence of nucleotide bases in DNA, RNA, rRNA, or any extracted genetic material. Additionally, sequencing may refer to reading and outputting the order sequence of amino acid residues from purified whole peptides, proteins, organic compounds, or polypeptides.

As used herein, the term “clustering” and grammatical variants thereof, refers generally to the grouping of genes and other biological sequences into categories based on shared characteristics including, but not limited to, ancestry, a shared generalized function, metabolic pathway, gene co-localization, gene co-expression, gene sequence similarity, the like, or any combination thereof. The process of clustering genes may be assisted with the use of pre-established algorithms or artificial intelligence.

As used herein, the term “biomarker,” “bio-marker,” “bio-indicator,” “molecular maker,” “signature marker,” and grammatical variants thereof, refers generally to a reproducible biological characteristic measured as: an indicator of a typical biological process, a response outside interference, a sign of a pathology, or the like. More specifically, monitoring bio-markers may assess the presence or progress of a disease or infection. In application at a hydrocarbon reservoir, bio-markers may detect: sources of spilled oil, the corrosion or degradation of the instruments on site, the quality of the hydrocarbon itself, or the like.

Collection of a sample may be carried out by any means necessary, and samples may be obtained at a plurality of time points. Samples may be collected in any amount. In non-limiting examples, a sample may be comprised in part of any sediment, soil, atmospheric dust and/or particles, water, or any liquid. Samples may be collected from typical hydrocarbon reservoir environments and equipment thereof. In non-limiting examples, samples may be collected from hydrocarbon reservoir production wells, pipes, pipelines, trunk lines, tubes, storage tanks, vesicles, corrosion failure paces, and any equipment used in the handling of petroleum, natural gas, chemicals, water, produced water, or any associated byproducts. In a non-limiting example, samples may originate from the immediate vicinity of a pipeline. “Immediate vicinity,” and grammatical variations thereof, as used herein refers to a three-dimensional (3D) space extending radially from any point on or within a pipeline, the 3D space extending radially from about 0.1 feet to about 300 feet. In non-limiting examples, samples may be collected in a plurality of methods as dictated in the NACE International Standard TM0212-2012.

The collected sample may have the protein present extracted and identified to determine the microorganisms producing them and establish protein bio-markers. Extraction, purification, and sequencing of protein or polypeptides from a collected sample may be carried out in any particular method, and in accordance with common practice that is familiar to those skilled in the art. In a non-limiting example, methods of sequencing of the amino acid residues of the collected and purified protein or polypeptides may include Edman degradation, next-generation protein sequencing (NGPS), mass spectrometry-based methods such as LC-MS, or any combination thereof.

Extraction and purification of genetic material from collected samples may be carried out in no particular method, and in accordance with common practice that is familiar to those skilled in the art. In a non-limiting embodiment, a sample may be purified with a DNA extraction kit such as an AQUADIEN™ DNA Extraction and Purification kit (available from Bio-Rad). A collected sample may first be filtered through a membrane and washed with a DNase-free water. The filter membrane may be a hydrophobic membrane, and may be comprised of a polycarbonate membrane, or the like. The quantity of a collected sample may range from about 100 μL to about 1 mL in volume. After filtration, the membrane may be placed in a tube and submerged in a lysis buffer solution and agitated (vortexed) to transfer the DNA from the filter into the lysis buffer solution. The lysis buffer solution supernatant may then be added to a purification column, centrifuged at an average speed range of about 6000 g for 10 minutes, and collected within the purification column while waste product is flowed through into a waste collection tube. Once centrifugation is complete, the purification column may then be moved into a clean collection tube, and an elution buffer may be added to the purification column to remove the DNA from the purification column. The purification column may again be centrifuged as the elution buffer removes the DNA from the purification column into the clean collection tube.

The goal of the genomic sequencing and analysis (of extracted DNA from a collected sample) is to produce genomic representative characters that correctly identify the microbial population within a sample. Sequencing and analysis of the extracted DNA from the collected sample may be carried out in any particular method, and may be conducted in accordance with common practice that is familiar to those skilled in the art. In a non-limiting embodiment, a target gene of a microbial organism within the extracted DNA may be first amplified before sequencing to identify organisms within the microbial population. Candidate target genes for amplification may include ribosomal genes such as 16S rRNA or 18S rRNA, or common housekeeping genes such as pmoA, mcrA, amoA, nirS, rpoB, nirK, pufM, nosZ, and any other conserved genetic markers. 16S rRNA amplicon is the standard amplification method for bacterial or archacal genomic identification due to the 9 variable regions (namely V2, V4, and V6) of the 16S rRNA gene that are highly abundant in bacterial and archaca genes. The 16S subunit is a bacteria and archaea ribosome gene, and is highly unique amongst species, while remaining unaffected by gene transfer.

After the amplification of the extracted DNA sequences based on marker gene(s), sequencing is done. In a non-limiting example, sequencing may comprise utilizing 1^{st} Generation Sanger Sequencing, or high-throughput Next Generation Sequencing (NGS) methods including, but not limited to, Illumina, Roche 454, PacBio, Oxford Tech IonTorrent, or any other methods of NGS.

To increase the resolution of sequenced metagenomes and assist in gene annotation, compiled reference databases of sequences may be used for comparison and accurate identification. In a non-limiting example, reference databases may comprise Greengenes, Ribosomal Database Project (RDP), SILVA ribosomal database, or any combination thereof. Comparative analysis and annotation with reference databases may be facilitated with comprehensive analysis servers such as MG-RAST.

Due to the low resolution of 16S amplicon and sequencing, alternative methods to amplifying and sequencing marker genes have arose in identifying microorganisms of a large population. In a non-limiting embodiment, the sequencing of extracted DNA may be conducted with metagenome shotgun whole genome sequencing (WGS). Briefly, this method randomly breaks down and sequences the entire genome of a plurality of organisms within a sample unrestricted, and reassembles overlapping sequences by computer program for individual sequencing. This yields a large data with a high resolution of the microbial population at the species level. Additionally, shotgun sequencing is a leading method in discovering new bacterial or archacal genes and genomes, and provides more specific taxonomic and functional classification of sequences in a sample. The output of shotgun sequencing may be trimmed with software to remove low quality data, and said software may comprise cutadapt, sickle, fastqMcf, or any software of the like.

The volume of data from shotgun WGS may require an extensive set of computational tools. Bioinformatics analysis of shotgun WGS output may include, but are not limited to, clade-specific maker genes, lowest common ancestor (LCA) taxonomy tree analysis, or the like. Analytical tools may further comprise, but not limited to, basic local alignment search tool (BLAST), ANalyzer, MEGAN5, MEtaGenome, MetaPhlAn, PhymmBL, Kraken, callisto, CLARK, PhyloSift, or any combination thereof.

After sequencing, an organism's taxa, proteins, and their associated metabolic byproducts may be identified and even predicted with the use of computational algorithms and artificial intelligence (AI) tools, and may be carried out in any particular method, and in accordance with common practice that is familiar to those skilled in the art. In addition to organism identification with aforementioned tools, protein signature, predicted protein function, and metabolic signatures may be recognized with computational algorithms and AI tools as well. In a non-limiting example, the identity of proteins present in the sample may be assessed with a sequence analysis tool such as InterProScan. The InterProScan Java-based application receives DNA or protein sequences from a subject, and analyzes them with predictive models trained on combined signatures from a plurality of disparate databases. The application clusters proteins into distinct domains and families, and labels predicted functional domains and conserved sequence sites within the protein sequence.

In a non-limiting example, the amino acid sequence of a protein may be read to predict the function of a sequenced proteins from a sample using a deep learning model such as the machine learning algorithm DeepGOPlus system. The DeepGOPlus system utilizes deep convolutional neural networks in addition to protein sequence similarity to make functional predictions. DeepGOPlus is first taught by the algorithm with a dataset based on MIC-related organisms. Once trained, the DeepGOPlus system is then fed the sequenced proteins from a sample to form functional predictions.

Clustering of sequenced and analyzed genomic data is focused on the task of classifying sequences into related groups according to a target process or compound, based on shared traits within the sequences. In a non-limiting example, clustering may group identified taxonomies based on function and or relative identity to a bacterial or archacal community directly or indirectly involved in the corrosion at hydrocarbon reservoir and associated operational equipment such as pipes, pipelines, trunklines, tubes, storage tanks, vesicles, and any equipment used in the handling of natural gas, chemicals, water, produced water, or any associated byproducts. Initial clustering may comprise sorting sequences de novo by grouping those with about 99.0% or greater sequence similarity, as well as clustering sequences by phenotype assessed by similarity to previous annotations from reference databases. In another non-limiting example, clustering may be conducted with a hierarchal clustering algorithm with a complete linkage method.

Once the plurality of sequenced microorganisms have been identified and clustered, the clustered sequences may be further sorted according to a functional relation to a process or compound. In a non-limiting example, these sorting categories (organism taxa, processes, compounds such as proteins or metabolites, or a combination thereof) may include proteins, enzymes, metabolites, metabolic byproducts, archaea species, bacteria species, eukaryotic species, or any combination thereof. Furthermore, these categories may be further sorted into subcategories in an unrestricted fashion.

In a non-limiting embodiment, sorting criteria may include microorganisms and their related byproducts responsible for or involved in the MIC or electrochemical corrosion of a hydrocarbon reservoir, and associated equipment such as pipes, pipelines, trunklines, tubes, storage tanks, vesicles, and any equipment used in the handling of natural gas, chemicals, water, produced water, or any associated byproducts. Methanogens, for example, are methane-producing archaca involved in MIC at the interface of metal surfaces or pipelines and serve as a relevant sorting category. More pervasive perpetrators of MIC, however, are sulfate reducing prokaryote (SRP) and sulfate reducing bacteria (SRB) found at corrosion sites at pipelines and would cast a wider net. SRP and SRB microorganisms may include, but are not limited to, archaca, metal reducing bacteria, iron reducing bacteria, fermentative bacteria, sulfate reducing bacteria, and sulfate reducing archaca. And due to ubiquity, metabolic byproducts of these SRP and SRB may also serve as crucial sorting categories. Using sulfate as a terminal electron acceptor, SRP and SRB may remove hydrogen at the cathode causing cathodic depolarization at metal surfaces and reducing sulfate to hydrogen sulfide (H₂S). H₂S may react with metals such as iron and steel, resulting in the formation of insoluble metal-sulfides, causing metals to soften or become pitted. Additionally, H₂S may acidify water leading to corrosion of carbon steel pipelines. Catalytic enzymes like hydrogenase may also serve as a sorting category, as the enzyme interacts with the metabolic byproduct H₂S to accelerate the corrosion via catalyzing the reversible oxidation of cathodic hydrogen.

Once sorting of the clustered data has confirmed established linkage to a process or compound, the common organism, trait, or molecule that distinguishes that sorted group may be characterized as a bio-marker. The biomarker may be used as an early indicator or warning sign of a biological process ongoing at the site of a collected sample. In a non-limiting example, the biomarker may signify a specific byproduct or enzymatic catalyst of the SRB lifecycle (such as H₂S or hydrogenase), or a bacteria or prokaryote that directly impacts electrode reaction kinetics. In a non-limiting example, bio-markers may be derived from crude oils, sediments, water, produced water, or rocks.

Embodiments of the present disclosure will now be described in detail with reference to the accompanying Figures. Like elements in the various figures may be denoted by like reference numerals for consistency. Additionally, it will be apparent to one of ordinary skill in the art that the scale of the elements presented in the accompanying Figures may vary without departing from scope of the present disclosure.

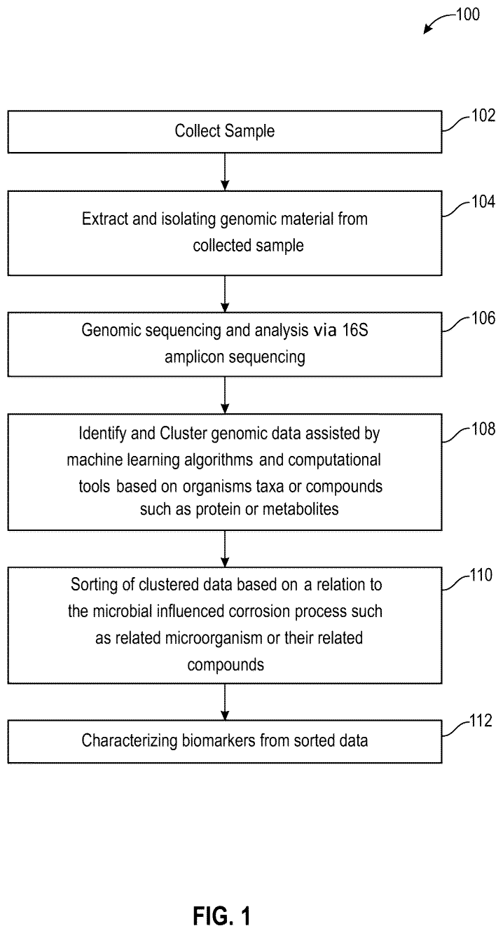

FIG. 1 is a block diagram of a non-limiting systems and methods of the present disclosure distinguishing microbial influenced corrosion (MIC) bio-markers within a mixed microbial population. At block 102, water, sediment, soil, atmospheric dust and/or particles, and any other liquid is collected from a hydrocarbon reservoir in a matter or technique that preserves the integrity of the sample. At block 104, genomic material is extracted and isolated from the collected sample, and further purified to remove any non-genomic material. The genomic material may be extracted by any standard DNA or RNA extraction techniques or protocols. Proteins may also be extracted from the collected sample and purified with any standard technique known to those of normal skill in the art.

At block 106, the purified genomic material is amplified and sequenced to provide genomic representative characters that correctly identify the microbial population within the collected sample. Amplification of the genomic sample may be carried out with 16S amplicon sequencing protocol to amplify the target gene on 16S microbial ribosomal rRNA gene. Sequencing of the amplified genomic sample is carried out by any sequencing standard technique known to those skilled in the art. Any protein or polypeptides extracted and purified from the collected sample may be sequenced by any method in accordance with common practice that is familiar to those skilled in the art. Sequencing of the collected sample would yield genomic data in the form of nucleotides or amino acids.

At block 108, the sequenced genomic data is identified and clustered by a machine learning (M.I.) algorithm and a plurality of computational tools. This clustering of genomic data is based on the taxa of the organisms identified in the sequenced genomic sample, compounds present in the sample such as proteins or metabolites, or function of proteins present in the genomic sample.

At block 110, the identified and clustered genomic samples are sorted on a basis of relation to the MIC process. This may include sorting categories such as an MIC-related organism or a MIC-related compound. At block 112, the aforementioned MIC sorted genomic data may be characterized as biomarker indicators of MIC within a collected sample.

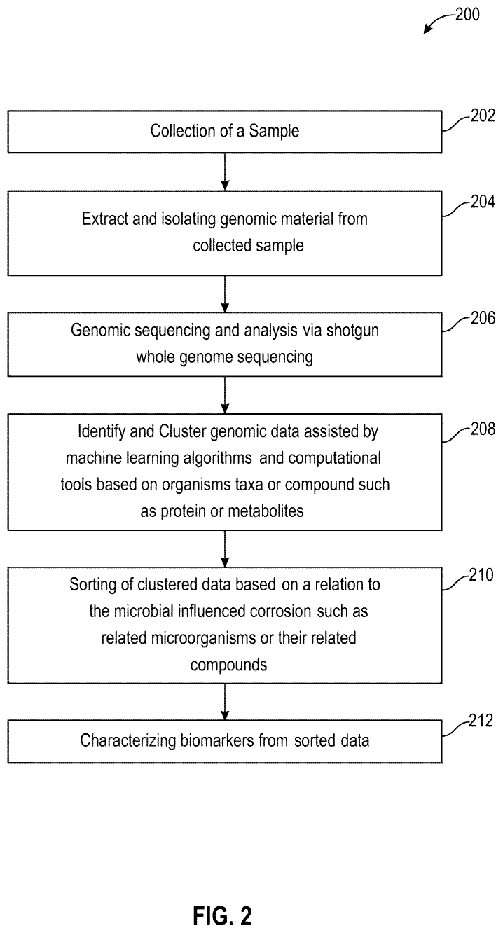

FIG. 2 is a is a block diagram of a non-limiting systems and method of the present disclosure for distinguishing microbial influenced corrosion (MIC) bio-markers within a mixed microbial population. The block diagram is similar to FIG. 1, with an alternative method of sequencing the collected genomic sample. At block 206, the collected genomic sample is sequenced and analyzed globally by shotgun whole genome sequencing, wherein the genomic sample is randomly broken down and sequenced as a whole, and computationally reassemble the overlapping sequences.

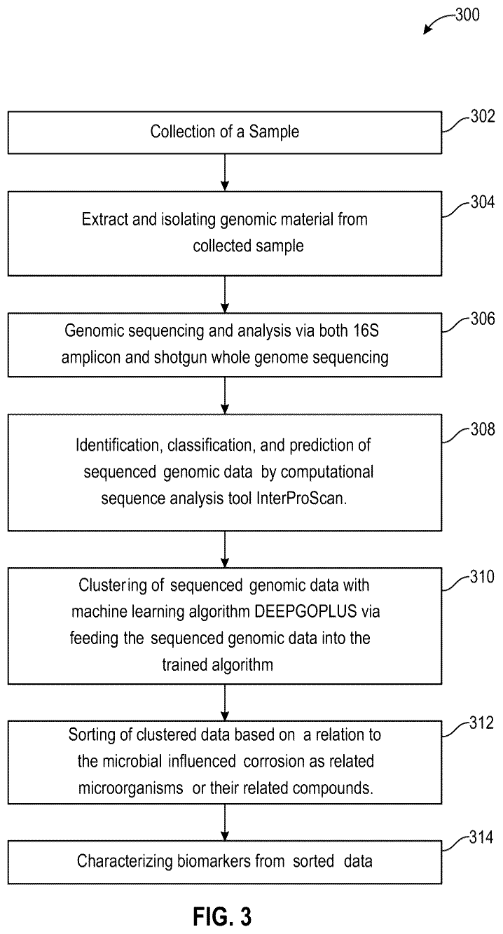

FIG. 3 is a block diagram of a non-limiting systems and method of the present disclosure distinguishing microbial influenced corrosion (MIC) bio-markers within a mixed microbial population. The block diagram is a system and method similar to FIG. 1, with deviations in the methods of sequencing the collected genomic sample, and the identification and clustering of the sequenced collected genomic sample. At block 306, sequencing and analysis of the purified genomic sample is carried out by both 16S amplicon sequencing and shotgun whole genome sequencing.

At block 308, the individual organisms, metabolites, and proteins present in the sequenced genomic sample are predicted, identified, and classified by computational sequence analytical tools. The computational sequence analysis tool for protein identification is InterProScan, where the Java-based application is fed DNA or protein sequences from the genomic sample and analyzed by predictive models.

At block 310, the identified sequenced genomic sample is clustered by a machine learning algorithm based an organisms taxa or relation to a compound by a protein or metabolites. The sequenced and identified genomic sample may be fed into the trained machine learning algorithm DEEPGOPLUS for clustering based on protein sequence similarity and make functional predictions.

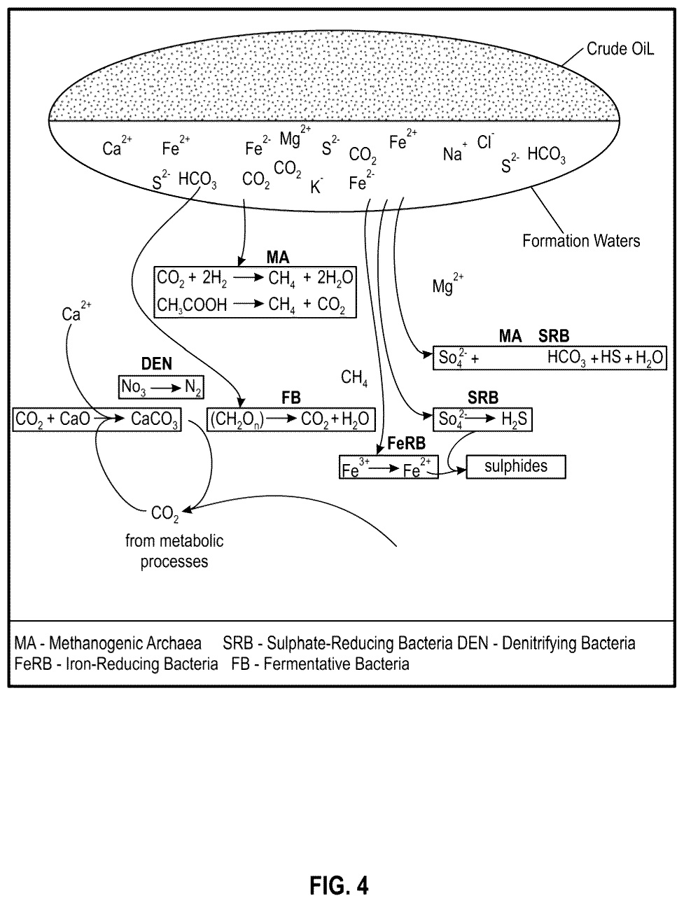

In a nonlimiting example, microorganisms that contribute to MIC may include methanogenic archaea, sulphate-reducing bacteria, denitrifying bacteria, iron-reducing bacteria, and fermentative bacteria, and the like. In a depiction not bound by theory, FIG. 4 shows the life cycle of microbes responsible for electrochemical corrosion in a hydrocarbon reservoir environment.

While various embodiments have been shown and described herein, modifications may be made by one skilled in the art without departing from the scope of the present disclosure. The embodiments described here are exemplary only, and are not intended to be limiting. Many variations, combinations, and modifications of the embodiments disclosed herein are possible and are within the scope of the disclosure. Accordingly, the scope of protection is not limited by the description set out above, but is defined by the claims which follow, that scope including all equivalents of the subject matter of the claims.

### Example Embodiments

Embodiments disclosed herein include:

Embodiment A: a method comprising: performing genomic analysis on a genomic material of a collected sample based on metagenomics sequencing to provide sequenced genomic data; clustering the sequenced genomic data to form sequenced genomic clusters; wherein the sequenced genomic clusters represent a predictive function provided by the sequenced genomic data to a trained machine learning (M.L.) predictive algorithm; sorting the sequenced genomic clusters into one or more microbial influenced corrosion-based categories, wherein the one or more microbial influenced corrosion-based categories are based on one or more microbial influenced corrosion-related microorganisms or a compound of the related microorganisms; and characterizing a corrosion biomarker of the one or more microbial influenced corrosion-based categories, wherein the corrosion biomarker is logically linked with an increased likelihood of microbial influenced corrosion based on the predictive function of the sequenced genomic clusters.

Embodiment A may have one or more of the following elements in any combination:
Element 1: wherein the collected sample originates from a hydrocarbon reservoir, and wherein the collected sample is collected by methods in accordance with the NACE International Standard TM0212-2012.
Element 2: wherein the characterized corrosion bio-markers identify the microbial consortia within the hydrocarbon reservoir.
Element 3: wherein the collected sample comprises water, bulk fluids, internal pipeline surfaces, or soil, wherein the water, the bulk fluids, the internal pipeline surfaces, or the soil originate from the immediate vicinity of a pipeline and may be selected from the group consisting of pipelines, trunk lines, tubes, storage tanks, vesicles, corrosion failure paces, and any combination thereof.
Element 4: wherein the compound is logically linked with hydrogen sulfate (H₂S).
Element 5: wherein the compound is logically linked with hydrogenase.
Element 6: wherein the microbial influenced corrosion-related microorganism is a methanogen.
Element 7: wherein the microbial influenced corrosion-related microorganism is a sulfate reducing prokaryote.
Element 8: wherein the sulfate reducing prokaryote comprises at least one selected from the group consisting of archaea, metal reducing bacteria, iron reducing bacteria, fermentative bacteria, sulfate reducing bacteria, sulfate reducing archaca, and any combination thereof.
Element 9: wherein the genomic material comprises ribosomal ribonucleic acid (rRNA).
Element 10: wherein the genomic analysis is conducted by 16S amplicon sequencing.
Element 11: wherein the whole metagenomics sequencing is performed by shotgun metagenomics sequencing for a plurality of genomic material in parallel.
Element 12: wherein the sequences of the genomic data are represented mathematically.
Element 13: wherein the sequence is associated with the taxonomic identification of the microorganism.
Element 14: wherein the compound is the metabolic products of the microorganism.
Element 15: wherein the clustering of genomic data is conducted with machine learning (M.L.)-based algorithms.
Element 16: wherein the clustering of genomic data is conducted with a protein signature algorithm.
Element 17: wherein the sorting of genomic data is conducted with a protein function prediction and annotation algorithm.
Element 18: wherein the corrosion biomarker dictates the use and frequency of a biocide treatment at the hydrocarbon reservoir.

Additional Embodiments disclosed herein include:

Embodiment 1. A method comprising: performing genomic analysis on a genomic material of a collected sample based on metagenomics sequencing to provide sequenced genomic data; clustering the sequenced genomic data to form sequenced genomic clusters; wherein the sequenced genomic clusters represent a predictive function provided by the sequenced genomic data to a trained machine learning (M.L.) predictive algorithm; sorting the sequenced genomic clusters into one or more microbial influenced corrosion-based categories, wherein the one or more microbial influenced corrosion-based categories are based on one or more microbial influenced corrosion-related microorganisms or a compound of the related microorganisms; and characterizing a corrosion biomarker of the one or more microbial influenced corrosion-based categories, wherein the corrosion biomarker is logically linked with an increased likelihood of microbial influenced corrosion based on the predictive function of the sequenced genomic clusters.

Embodiment 2. The method of embodiment 1, wherein the collected sample originates from a hydrocarbon reservoir, and wherein the collected sample is collected by methods in accordance with the NACE International Standard TM0212-2012.

Embodiment 3. The method of embodiment 1 or 2, wherein the characterized corrosion bio-markers identify the microbial consortia within the hydrocarbon reservoir.

Embodiment 4. The method of embodiment 1-3, wherein the collected sample comprises water, bulk fluids, internal pipeline surfaces, or soil, wherein the water, the bulk fluids, the internal pipeline surfaces, or the soil originate from the immediate vicinity of a pipeline and may be selected from the group consisting of pipelines, trunk lines, tubes, storage tanks, vesicles, corrosion failure paces, and any combination thereof.

Embodiment 5. The method of embodiment 1-4, wherein the compound is logically linked with hydrogen sulfate (H₂S).

Embodiment 6. The method of embodiment 1-5, wherein the compound is logically linked with hydrogenase.

Embodiment 7. The method of embodiment 1-6, wherein the microbial influenced corrosion-related microorganism is a methanogen.

Embodiment 8. The method of embodiment 1-7, wherein the microbial influenced corrosion-related microorganism is a sulfate reducing prokaryote.

Embodiment 9. The method of embodiment 1-8, wherein the sulfate reducing prokaryote comprises at least one selected from the group consisting of archaea, metal reducing bacteria, iron reducing bacteria, fermentative bacteria, sulfate reducing bacteria, sulfate reducing archaca, and any combination thereof.

Embodiment 10. The method of embodiment 1-9, wherein the genomic material comprises ribosomal ribonucleic acid (rRNA).

Embodiment 11. The method of embodiment 1-10, wherein the genomic analysis is conducted by 16S amplicon sequencing.

Embodiment 12. The method of embodiment 1-11, wherein the whole metagenomics sequencing is performed by shotgun metagenomics sequencing for a plurality of genomic material in parallel.

Embodiment 13. The method of embodiment 1-12, wherein the sequences of the genomic data are represented mathematically.

Embodiment 14. The method of embodiment 1-13, wherein the sequence is associated with the taxonomic identification of the microorganism.

Embodiment 15. The method of embodiment 1-14, wherein the compound is the metabolic products of the microorganism.

Embodiment 16. The method of embodiment 1-15, wherein the clustering of genomic data is conducted with machine learning (M.L.)-based algorithms.

Embodiment 17. The method of embodiment 1-16, wherein the clustering of genomic data is conducted with a protein signature algorithm.

Embodiment 18. The method of embodiment 1-17, wherein the sorting of genomic data is conducted with a protein function prediction and annotation algorithm.

Embodiment 19. The method of embodiment 1-18, wherein the corrosion biomarker dictates the use and frequency of a biocide treatment at the hydrocarbon reservoir.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

1. A method comprising:
performing genomic analysis on a genomic material of a collected sample based on metagenomics sequencing to provide sequenced genomic data;
clustering the sequenced genomic data to form sequenced genomic clusters;
wherein the sequenced genomic clusters represent a predictive function provided by the sequenced genomic data to a trained machine learning (M.L.) predictive algorithm;
sorting the sequenced genomic clusters into one or more microbial influenced corrosion-based categories, wherein the one or more microbial influenced corrosion-based categories are based on one or more microbial influenced corrosion-related microorganisms or a compound of the related microorganisms; and
characterizing a corrosion biomarker of the one or more microbial influenced corrosion-based categories, wherein the corrosion biomarker is logically linked with an increased likelihood of microbial influenced corrosion based on the predictive function of the sequenced genomic clusters.

2. The method of claim 1, wherein the collected sample originates from a hydrocarbon reservoir, and wherein the collected sample is collected by methods in accordance with the NACE International Standard TM0212-2012.

3. The method of claim 2, wherein the characterized corrosion bio-markers identify the microbial consortia within the hydrocarbon reservoir.

4. The method of claim 1, wherein the collected sample comprises water, bulk fluids, internal pipeline surfaces, or soil, wherein the water, the bulk fluids, the internal pipeline surfaces, or the soil originate from the immediate vicinity of a pipeline and may be selected from the group consisting of pipelines, trunk lines, tubes, storage tanks, vesicles, corrosion failure paces, and any combination thereof.

5. The method of claim 1, wherein the compound is logically linked with hydrogen sulfate (H₂S).

6. The method of claim 1, wherein the compound is logically linked with hydrogenase.

7. The method of claim 1, wherein the microbial influenced corrosion-related microorganism is a methanogen.

8. The method of claim 1, wherein the microbial influenced corrosion-related microorganism is a sulfate reducing prokaryote.

9. The method of claim 8, wherein the sulfate reducing prokaryote comprises at least one selected from the group consisting of archaea, metal reducing bacteria, iron reducing bacteria, fermentative bacteria, sulfate reducing bacteria, sulfate reducing archaea, and any combination thereof.

10. The method of claim 1, wherein the genomic material comprises ribosomal ribonucleic acid (rRNA).

11. The method of claim 1, wherein the genomic analysis is conducted by 16S amplicon sequencing.

12. The method of claim 1, wherein the whole metagenomics sequencing is performed by shotgun metagenomics sequencing for a plurality of genomic material in parallel.

13. The method of claim 1, wherein the sequences of the genomic data are represented mathematically.

14. The method of claim 1, wherein the sequence is associated with the taxonomic identification of the microorganism.

15. The method of claim 1, wherein the compound is the metabolic products of the microorganism.

16. The method of claim 1, wherein the clustering of genomic data is conducted with machine learning (M.L.)-based algorithms.

17. The method of claim 16, wherein the clustering of genomic data is conducted with a protein signature algorithm.

18. The method of claim 1, wherein the sorting of genomic data is conducted with a protein function prediction and annotation algorithm.

19. The method of claim 2, wherein the corrosion biomarker dictates the use and frequency of a biocide treatment at the hydrocarbon reservoir.
